(19) United States
(12) Patent Application Publication
(10) Pub. No.: US 2026/0192099 A1
(43) Pub. Date: Jul. 9, 2026

(54) APPLICATOR WITH TINTING AGENT

(71) Applicant: CareFusion 2200, Inc., San Diego, CA (US)

(72) Inventors: Angel G. MAGALLON, San Diego, CA (US); James Patrick McDONALD, San Diego, CA (US)

(21) Appl. No.: 19/130,388

(22) PCT Filed: Nov. 15, 2023
(86) PCT No.: PCT/US2023/079783

§ 371 Date: May 15, 2025

Related U.S. Application Data
(60) Provisional application No. 63/383,826, filed on Nov. 15, 2022.

(51) Int. Cl. A61M 35/00

(52) U.S. Cl. CPC A61M 35/006

(57) ABSTRACT

Disclosed herein is an applicator having a body portion housing a fluid and an application member having a tinting agent releasably immobilized therein and/or thereon, wherein the applicator is free of tinting agent within the body portion. Also disclosed are methods of making the same.

### CROSS-REFERENCE TO RELATED APPLICATIONS

This application claims priority to U.S. Provisional Application No. 63/383,826, filed on Nov. 15, 2022, the contents of which are expressly incorporated herein in their entirety.

### TECHNICAL FIELD

The present disclosure relates to applicators configured to provide a tinted cleaning fluid.

### BACKGROUND

Applicators are frequently used in medical settings, for example, to provide a cleaning fluid to a patient and/or a device prior to a medical procedure. In such instances, it is often beneficial for the cleaning fluid to be tinted such that a user can easily visualize the application of the cleaning fluid.

Previously, applicators have been provided with a component such as a pledget within the body of the applicator, the pledget containing a dye capable of tinting a cleaning fluid as the cleaning fluid passes therethrough. However, these pledgets are associated with several disadvantages. For example, the use of such pledgets often results in dye being inadvertently released within the applicator during assembly.

In addition, known processes for providing dye to applicator components (e.g., to a pledget) are associated with several disadvantages. For example, processes known as “dunk and dry” processes, wherein materials are submerged in a dye and allowed to dry, generally result in applicator components showing inconsistent dye outputs. In addition, such processes are often time-consuming and consume excess dye (i.e., an amount of dye beyond the amount needed to tint a cleaning fluid).

There thus remains a need for applicators that avoid problems associated with current applicators as known in the art.

### SUMMARY

The present disclosure is directed to an applicator having a body portion containing a fluid, and an application member having a tinting agent releasably immobilized therein and/or thereon, wherein the applicator is free of tinting agent within the body portion.

The present disclosure is also directed to methods of making an application member as described herein, including providing an application member material and applying a tinting agent to the application member material via a controllable tinting process sufficient to releasably immobilize the tinting agent on and/or within the application member material.

### BRIEF DESCRIPTION OF THE DRAWINGS

FIG. 1 shows an example applicator according to aspects of the present disclosure.

FIG. 2A shows an example application member according to aspects of the present disclosure.

FIG. 2B shows an example application member according to aspects of the present disclosure.

FIG. 2C shows an example application member according to aspects of the present disclosure.

FIG. 2D shows an example application member according to aspects of the present disclosure.

FIG. 2E shows an example application member according to aspects of the present disclosure.

FIG. 3 shows an example applicator according to aspects of the present disclosure.

### DETAILED DESCRIPTION

The present disclosure is directed to an applicator having a body portion and an application member, wherein the application member is configured to releasably immobilize a tinting agent. The tinting agent according to the present disclosure may be configured to tint a fluid prior to and/or during application of the fluid to a surface by the application member. According to some aspects, the applicator of the present disclosure may be free of tinting agent within the body portion.

The present disclosure is also directed to methods of making the applicator or portions thereof, such as the application member. According to some aspects, the method may include providing an application member material and applying a first amount of tinting agent to the application member material via a controllable tinting process.

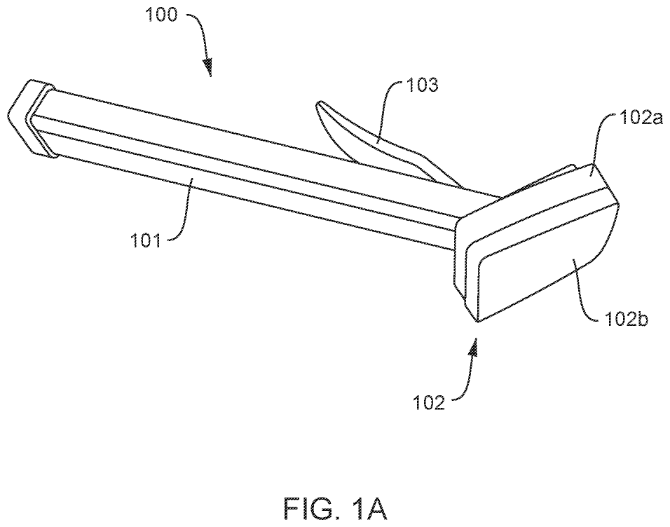

FIG. 1 shows an example applicator as described herein. As shown in FIG. 1, applicator 100 has a body portion 101 and an application member 102. According to some aspects, body portion 101 may be configured to house a fluid to be applied to a surface via applicator 100. For example, body portion 101 may be configured to house one or more ampoules and/or similar containers (not shown) in which a fluid may be contained prior to application of the fluid to a surface. Additionally or alternatively, the body portion may itself house a fluid prior to application of the fluid to a surface (not shown). Applicator 100 may optionally include an actuator 103 configured to actuate applicator 100, wherein actuation of applicator 100 corresponds to body 101 being provided in fluid communication with application member 102 via a fluid path (not shown). In some non-limiting examples, actuation of actuator 103 may comprise fracturing, puncturing, shattering, and/or otherwise breaching an ampoule such that fluid contained within the ampoule is released.

Although not shown, applicator 100 may further include a fluid metering device, such as a pledget. According to some aspects, the fluid metering device may be provided within body portion 101 in order to control and/or direct the flow of fluid from body portion 101 to application member 102 described herein.

Non-limiting example applicators that may be used according to the present disclosure may be found, for example, in Applicant's U.S. Pat. Nos. 5,690,958; 6,536,975; 7,993,066; 8,708,983; 8,899,859; 9,119,946; 9,572,967; 9,757,551; 9,968,764; 10,076,648; and 10,813,892, the disclosures of which are incorporated herein by reference in their entirety. According to some aspects, the applicator may be configured to house and/or apply a selected amount of fluid, alternatively referred to herein as an “applicator size.” In some non-limiting examples, the applicator size may be about 0.5 mL, about 1 mL, about 1.5 mL, about 2 mL, about 2.5 mL, about 3 mL, about 3.5 mL, about 4 mL, about 4.5 mL, about 5 mL, about 5.5 mL, about 6 mL, about 6.5 mL, about 7 mL, about 7.5 mL, about 8 mL, about 8.5 mL, about 9 mL, about 9.5 mL, about 10 mL, about 10.5 mL, about 11 mL, about 11.5 mL, about 12 mL, about 12.5 mL, about 13 mL, about 13.5 mL, about 14 mL, about 14.5 mL, about 15 mL, about 15.5 mL, about 16 mL, about 16.5 mL, about 17 mL, about 17.5 mL, about 18 mL, about 18.5 mL, about 19 mL, about 19.5 mL, about 20 mL, about 20.5 mL, about 21 mL, about 21.5 mL, about 22 mL, about 22.5 mL, about 23 mL, about 23.5 mL, about 24 mL, about 24.5 mL, about 25 mL, about 25.5 mL, or about 26 mL, as known in the art.

In some non-limiting examples, the fluid as described herein may include a cleaning fluid, such as a biocide, a biostat, or combinations thereof. As used herein, the term “biocide” refers to a chemical agent that inactivates microorganisms. As used herein, the term “biostat” refers to a chemical agent that reduces and/or prevents the growth of microorganisms. It should be understood that in some instances, a chemical agent may function as a biocide and a biostat.

Example biocides and/or biostats according to the present disclosure include antibiotics, antiseptics, and disinfectants. As used herein, an “antibiotic” is a naturally occurring or synthetic organic substance which inhibits or destroys selective bacteria or other microorganisms, generally at low concentrations. As used herein, an “antiseptic” is a biocide and/or biostat that destroys or inhibits the growth of microorganisms in or on living tissue. As used herein, a “disinfectant” is a biocide and/or biostat that destroys or inhibits the growth of microorganisms in or on an inanimate surface.

Non-limiting examples of biocides and/or biostats according to the present disclosure include alcohols, aldehydes, anilides, biguanides, diamidines, halogen-releasing agents, silver compounds, peroxygens, phenols, bis-phenols, halophenols, quaternary ammonium compounds, combinations thereof, and solutions thereof.

According to some aspects, the cleaning fluid may be a cleaning solution include one or more biocides and/or biostats and a solvent. For example, the cleaning fluid may be an antiseptic solution having an antiseptic and a solvent. According to some aspects, the cleaning solution is an aqueous solution. As used herein, the term “aqueous solution” refers to a solution wherein the solvent comprises at least a majority of water. According to some aspects, the cleaning solution is an organic solution. As used herein, the term “organic solution” refers to a solution wherein the solvent comprises at least a majority of an organic component, such as an alcohol.

According to some aspects, the antiseptic may include a cationic molecule (i.e., a molecule having a positive charge), such as a cationic surfactant or a cationic biguanide derivative (i.e., a compound derived from biguanide). According to some aspects, the antiseptic may include a bis-(dihydropyridinyl)-decane derivative (i.e., a compound derived from bis-(dihydropyridinyl)-decane). According to some aspects, the antiseptic may include an octenidine salt and/or a chlorhexidine salt. Non-limiting examples of antiseptics useful according to the present disclosure include octenidine dihydrochloride, chlorhexidine gluconate, povidone iodine, alcohols, and combinations thereof.

According to some aspects, the concentration of each biocide and/or biostat in the cleaning solution, or alternatively the total concentration of biocides and/or biostats in the cleaning solution, may be from about 0.0001% to about 2.0% w/v, optionally from about 0.01% to about 1% w/v, optionally from about 0.1% to about 0.4% w/v. According to some aspects, the concentration of each biocide and/or biostat in the cleaning solution, or alternatively the total concentration of biocides and/or biostats in the cleaning solution, may be from about 0.0001% to about 0.4% w/v, and optionally from about 0.1% to about 0.2% w/v. According to some aspects, the concentration of each biocide and/or biostat in the cleaning solution, or alternatively the total concentration of biocides and/or biostats in the cleaning solution, may be from about 0.5% to about 3.5% w/v, optionally from about from about 0.5% to about 2.0% w/v, and optionally about 2.0% w/v.

According to some aspects, the solvent may include an organic solvent, such as an alcohol, an organosulfur compound, a ketone, or a combination thereof. Non-limiting examples of alcohols include methanol, ethanol, propanol, such as n-propanol and/or isopropanol, and combinations thereof. One non-limiting example of a ketone includes acetone. One non-limiting example of an organosulfur compound includes dimethyl sulfoxide (DMSO). According to some aspects, the concentration of organic solvent in the cleaning solution may be from about 50% to about 90% v/v, optionally from about 70% to about 80% v/v, and optionally about 70% v/v. According to some aspects, the concentration of alcohol in the cleaning solution may be from about 10% to about 50% v/v, and optionally from about 20% to about 30% v/v.

According to some aspects, the solvent may include water. According to some aspects, the concentration of water in the cleaning solution may be from about 10% to about 50% v/v, and optionally from about 20% to about 30% v/v. According to some aspects, the concentration of water in the cleaning solution may be from about 50% to about 90% v/v, and optionally from about 70 to about 80% v/v.

According to some aspects, the cleaning solution may further include one or more film-forming polymers. Non-limiting examples of film-forming polymers include acrylate polymers (such as acrylamide polymers, octylacrylamide polymers, methacrylate polymers), carboxyacrylate polymers, carboxylate acrylic copolymers, acrylate copolymers, polymethacrylate-based co-polymers, hydroxypropyl cellulose, and polymers having dimethylaminoethyl methacrylate, butyl methacrylate, and methyl methacrylate side groups. The concentration of film-forming polymer may be varied depending on the particular solvent and biocide and/or biostat present in the cleaning solution.

According to some aspects, the concentration of film-forming polymer in the cleaning solution may be from about 0.1% to about 5% w/v, optionally from about 0.2% to about 3% w/v, optionally from about 0.5% to about 2.0% w/v, and optionally from about 0.75% to about 2.5% w/v.

Example acrylate polymers include, but are not limited to, DERMACRYL® AQF (2-propenoic acid, 2-methyl-, polymer with butyl 2-propenoate and methyl 2-methyl-2-propenoate), DERMACRYL® 79 P (2-propenoic acid, 2-methyl-, 2-methylpropyl ester, polymer with 2-propenoic acid and N-(1,1,3,3tetramethylbutyl)-2-propenamide), each manufactured by Akzo Nobel Coatings Inc, and EUDRAGIT® E PO (poly(butyl methacylate-co-(2-dimethylaminoethyl) methacrylate-co-methyl methacrylate) manufactured by Evonik Industries. DERMACRYL® 79P is a hydrophobic, high molecular weight carboxylated acrylic copolymer. EUDRAGIT® E PO is a cationic copolymer based on dimethylaminoethyl methacrylate, butyl methacrylate, and methyl methacrylate.

According to some aspects, application member 102 of the present disclosure may include a material sufficient for releasably immobilizing a tinting agent. As used herein, a tinting agent is “releasably immobilized” if at least a portion of the tinting agent molecules are immobilized by application member 102 prior to one or more events and at least a portion of the tinting agent molecules are released in response to the one or more events.

In one non-limiting example, the event may be contact between the tinting agent and one or more components of a cleaning fluid as described herein. In this example, the tinting agent may be releasably immobilized by application member 102 until a cleaning fluid is provided to application member 102, for example, via actuation of applicator 100 as described herein. In this example, at least a portion of the tinting agent molecules provided by the tinting agent may be released by application member 102 sufficient to tint a cleaning fluid passing through application member 102.

In another non-limiting example, the event may be a selected cumulative scrubbing action provided to application member 102. In this example, the tinting agent may be releasably immobilized by application member 102 until a selected cumulative scrubbing action is provided to application member 102, for example, by a surface to which application member 102 is to apply a cleaning fluid. In this example, when the selected cumulative scrubbing action has been achieved, at least a portion of the tinting agent molecules provided by the tinting agent may be released by application member 102 sufficient to tint a cleaning fluid passing through application member 102.

In another non-limiting example, the event may be a certain temperature. In this example, the tinting agent may be releasably immobilized by application member 102 until application member 102 reaches a selected temperature. The selected temperature may be obtained by providing heat to application member 102, such as the heat provided by a surface (e.g., a subject's skin), the heat provided by friction (e.g., friction between application member 102 and a surface), or a combination thereof. In some non-limiting examples, the selected temperature may be between about 25 and 40° C., optionally between about 25 and 35° C., optionally between about 35 and 40° C., optionally between about 33 and 37° C., optionally about 30° C., optionally about 31° C., optionally about 32° C., optionally about 33° C., optionally about 34° C., optionally about 35° C., optionally about 36° C., optionally about 37° C., optionally about 38° C., optionally about 39° C., and optionally about 40° C.

According to some aspects, the tinting agent may be releasably immobilized by application member 102 via any chemical and/or physical interaction sufficient to releasably immobilize the tinting agent as described herein. Non-limiting examples of interactions include adsorption, absorption, and combinations thereof. According to some aspects, all or a portion of the tinting agent may be releasably immobilized on a surface of application member 102, on an interior portion of application member 102 (e.g., by entrapment within a pore, pocket, etc.), or a combination thereof.

In some non-limiting examples, the tinting agent may include one or more anionic tinting agents, such as one or more anionic dyes. The anionic dye(s) may be any dye suitable for medical use, such as dyes approved by the Food and Drug Administration for use in food, drugs, and/or cosmetics (i.e., “D&C” or “FD&C” dyes). Example anionic dyes include, but are not limited to, FD&C Blue No. 1 (Brilliant Blue FCF), FD&C Blue No.2 (Indigo Carmine), FD&C Green No. 3 (Fast Green FCF), FD&C Red No. 3 (Erythrosine), FD&C Red No. 40 (Allura Red), FD&C Yellow No. 5 (Tartrazine), FD&C Yellow No. 6 (Sunset Yellow FCF), D&C Yellow No. 8 (Fluorescein), D&C Orange No. 4, and combinations thereof. Combinations may be implemented to arrive at a particular color. For example, an orange tint may comprise both FD&C Red No. 40 and D&C Yellow No. 8. According to some aspects, the tinting agent may be provided as part of a signaling element, for example, as described in Applicant's U.S. Pat. No. 10,982,980, the contents of which are explicitly incorporated by reference herein in their entirety.

According to some aspects, application member 102 may include an application member material sufficient for releasably immobilizing a tinting agent as described herein. Non-limiting examples of application member materials include foams, felts, nonwoven materials, and combination thereof. In some examples, all or a portion of the application member materials may be hydrophobic or hydrophilic. Non-limiting examples of materials useful for the application member material include those described in U.S. Pat. No. 7,993,066, the contents of which are incorporated by reference herein. For example, the application member material may include an open-celled foam material, such as a hydrophilic or hydrophobic polyester-polyurethane foam. Additionally or alternatively, the application member material may include a nonwoven material.

According to some aspects, application member 102 may have one, two, three, four, or more discrete layers, wherein each of the discrete layers individually include the same or different application member material as the application member material included by another discrete layer. According to some aspects, all of the discrete layers may releasably immobilize a tinting agent as described here, wherein each of the discrete layers releasably immobilizes the same tinting agent as or a different tinting agent from the tinting agent releasably immobilized by at least one other discrete layer. Alternatively, one or more of the discrete layers may be free of a tinting agent as described herein.

FIG. 1 shows one non-limiting example of an application member 102 having a first layer 102a that is proximal to body portion 101 and a second layer 102b that is distal to body portion 101. In one example, both layers 102a, 102b may releasably immobilize a tinting agent. Alternatively, only one of layers 102a, 102b may releasably immobilize the tinting agent. For example, layer 102a may include a nonwoven material that does not releasably immobilize a tinting agent (or is otherwise free of tinting agent), and layer 102b may include a foam, wherein at least a portion of the foam releasably immobilizes a tinting agent as described herein.

It should be understood that applicator as described herein is not limited to the example shown in FIG. 1. For example, application member 102 may have only one layer. Alternatively, application member 102 may have three, four, or more layers as described herein.

According to some aspects, at least one layer of application member 102 may have a tinting portion wherein tinting agent is releasably immobilized. According to some aspects, one layer of application member 102 may have a tinting portion wherein tinting agent is releasably immobilized, and another layer of application member 102 is free of tinting agent. According to some aspects, application member 102 may have a non-tinting portion that is free of tinting agent.

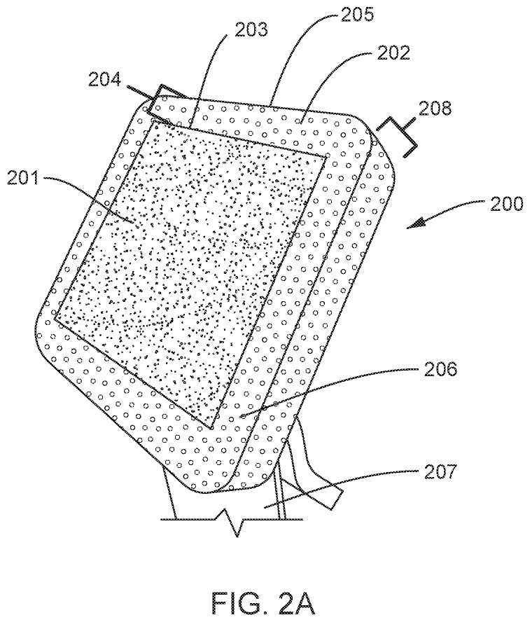

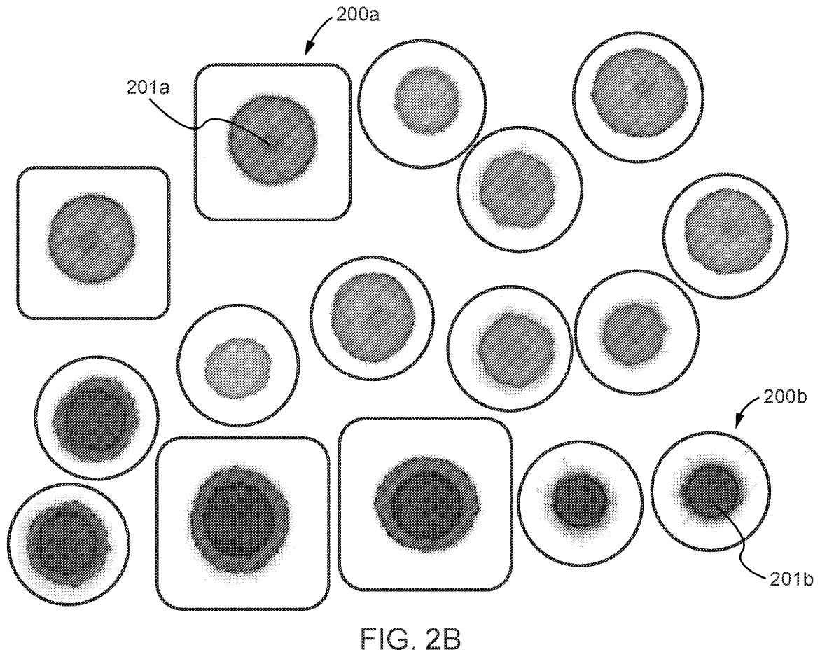

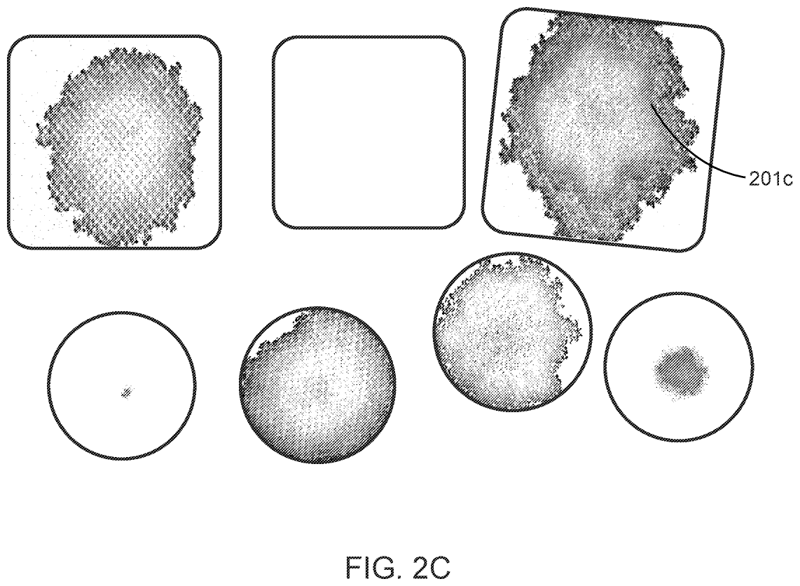

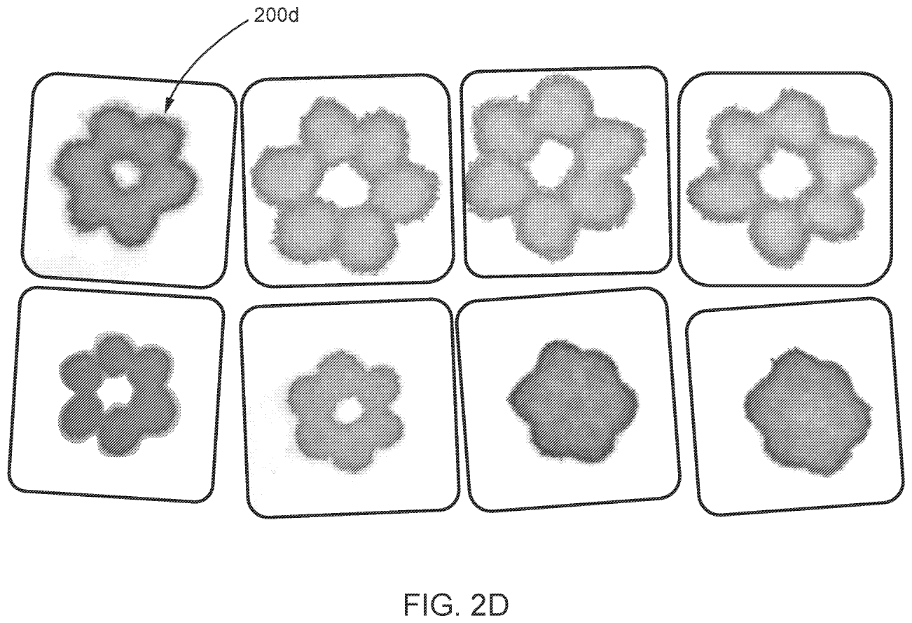

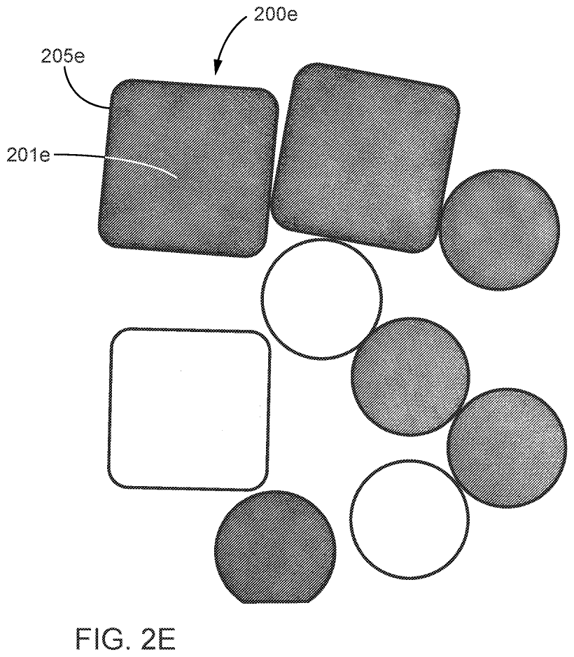

For example, FIG. 2A shows a photograph of an application member 200 having a tinting portion 201 and a non-tinting portion 202. According to some aspects, tinting portion 201 may be defined by one or more interior boundaries 203 that are spaced a distance 204 from an outer edge 205 of application member 200. According to some aspects, distance 204 may be sufficient to reduce or eliminate bleed of tinting agent from application member 200 during production and/or storage of the application member. For example, distance 204 may be sufficient to reduce or eliminate bleed of tinting agent from application member 200 during a terminal sterilization process. Additionally or alternatively, distance 204 may be sufficient for reduce or eliminate bleed of tinting agent from application member 200 when an applicator is provided in a packaging, as known in the art.

According to some aspects, tinting portion 201 may be defined by one or more interior boundaries 203 that are individually between about 0 to 0.3 inches from the closest outer edge 205 of application member 200. In some non-limiting examples, one or more outer edges of the application member may be free of tinting agent. In some examples, all of the outer edges of the application member are free of tinting agent.

According to some aspects, tinting portion 201 may have a certain depth. That is, molecules of tinting agent may penetrate application member 200 a certain distance from a surface 206 of application member 200, the certain distance being relative to a thickness 208 of application member 200. As used herein, the “thickness” of an application member is the dimension of an application member that extends from a distal to a proximal end relative to an applicator body provided therewith.

According to some aspects, tinting portion 201 may have a depth that is between about 0.01 and 100% of the total application member thickness, optionally between about 0.01 and 90%, optionally between about 0.01 and 80%, optionally between about 0.01 and 70%, optionally between about 0.01 and 60%, optionally between about 0.01 and 50%, optionally between about 0.01 and 40%, optionally between about 0.01 and 30%, optionally between about 0.01 and 20%, and optionally between about 0.01 and 10%.

It should be understood that while FIG. 2A shows tinting portion 201 extending a certain distance from surface 206 of application member 200 (surface 206 being distal to an applicator body 207 provided therewith), the application member of the present disclosure is not necessarily limited in this way. For example, the surface may be a surface proximal an applicator body, a surface between one or more layers of the application member, or any combination thereof. It should also be understood that while FIG. 2A shows a generally square application member 200 have a generally square tinting portion 201, the present disclosure is not limited in this way. For example, application member 200 and/or tinting portion 201 may have any other shape as may be useful in the art, such as a polygonal shape, a circular shape, and/or an ovular shape. For example, FIG. 2B shows a generally square application member 200a having a generally circular tinting portion 201a. FIG. 2B also shows a generally circular application member 200b having a generally circular tinting portion 201b. In some non-limiting examples, the application member and/or the tinting portion may have an irregular shape, such as irregularly shaped tinting portion 201c shown in FIG. 2C. In another example, FIG. 2D shows a tinting portion 201d having a “flower shape.” In addition, the tinting portion may comprise a continuous region, or it may comprise more than one discrete region, and may be comprised of shapes such as stripes, dots, triangles, etc. It should be understood that any shaped application member and tinting portion as described herein, and any combination thereof, is encompassed by the present disclosure. In addition, it should be understood that one or more boundaries of the tinting portion may extend to the closest outer edge of the application member such that there is no distance therebetween. For example, FIG. 2E shows an application member 200e having a tinting portion 201e that covers an entire face thereof such that there is no distance between a boundary of tinting portion 201e and an outer edge 205e of application member 200e.

According to some aspects, the applicator of the present disclosure may be free of tinting agent within the body portion. For example, the fluid metering device of the applicator may be free of tinting agent as described herein.

According to some aspects, tinting portion 201 may be provided by a controllable tinting process. As used herein, a “controllable tinting process” refers to a process for providing tinting agent to a material (e.g., an application member material) that allows for a selectable amount of tinting agent to be releasably immobilized and/or that allows for a selectable configuration (e.g., a shape and/or depth) of a resulting tinting portion as described herein.

In some non-limiting examples, the application member of the present disclosure may releasably immobilize between about 0.001 and 50 mg of tinting agent, optionally between about 0.001 and 30 mg, and optionally between about 2 and 30 mg. In some non-limiting examples, the application member may releasably immobilize between about 0.001 and 10 mg of tinting agent, optionally between about 0.001 and 1 mg, and optionally between about 0.01 and 0.15 mg. It should be understood that the amount of tinting agent immobilized by an application member may depend on the identity of the tinting agent and/or application member material. For example, the application member may include between about 1 and 50 mg, optionally between about 6 and 50 mg, and optionally between about 2 and 30 mg of FD&C Yellow 6 and/or between about 0.01 and 0.45 mg, optionally between about 3 and 0.45 mg, and optionally between about 0.01 and 0.15 mg for FD&C Green 3. The amount of tinting agent may depend on certain characteristics of the tinting agent, for example, the visibility of the tinting agent on a desired surface (e.g., the skin). The controllable tinting process of the present disclosure may therefore also be adjustable such that the selected amount of tinting agent and/or selected configuration of resulting tinting portion may be varied.

According to some aspects, the controllable tinting process may include a spraying step wherein tinting agent is sprayed onto an application member material as described herein. As used herein, the term “spray” refers to the deposition of an aerosolized material (e.g., an aerosolized tinting agent). In some non-limiting examples, the spraying step may include an electrostatic spraying step. According to some aspects, the aerosolized material may include a tinting agent and optionally a solvent as described herein. For example, the solvent may include water, an alcohol, or a combination thereof. Additionally or alternatively, the controllable tinting process may include an injection step, a coating step (including a roll-on coating step), a dipping step, or a combination thereof, wherein a tinting agent is applied to the application member material via injection, coating, and/or dipping, respectively. In these examples, the tinting agent may optionally be provided as a solution having a solvent as described herein. For example, the solvent may include water, an alcohol, or a combination thereof.

According to some aspects, the controllable tinting process may further include a drying step after the spraying step, the injection step, the coating step, and/or the dipping step. The drying step may include drying the application member material by air, forced convection, heated forced convection, an external heat source, or a combination thereof, simultaneously and/or sequentially. According to some aspects, the drying step may include drying the application member material to room temperature. As used herein, room temperature refers to a temperature between about 20 and 22° C.

In some non-limiting examples, tinting agent may be provided to the application member material by a controllable tinting process before, during, or after the manufacture of an application member from the application member material. For example, an application member material may be provided with the tinting agent and subsequently processed to form an application member (e.g., by cutting or otherwise dividing the application member material and/or attaching a portion of the application member material to a body portion of an applicator as described herein). Additionally or alternatively, an application member material may be provided with the tinting agent after the application member material has been cut or otherwise divided and/or attached to a body portion of an applicator.

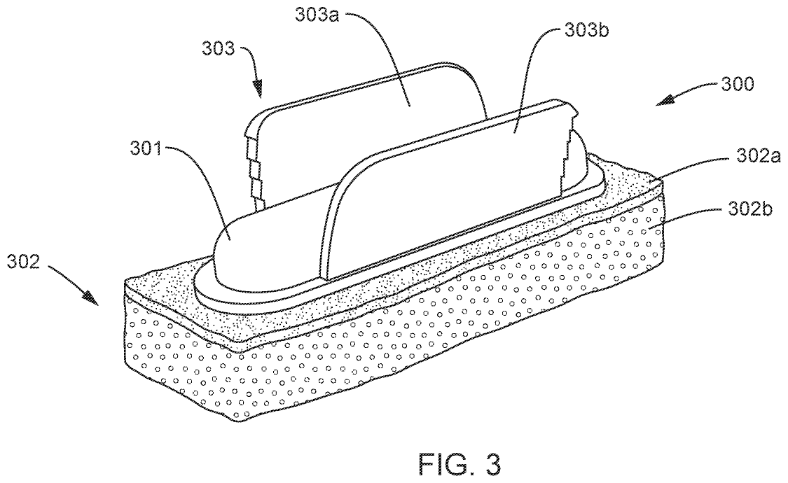

FIG. 3 shows another example of an applicator according to the present disclosure. As shown in FIG. 3, applicator 300 may include a body portion 301 and an applicator member 302, similar to those described in relation to FIG. 1. Application member 302 may include first layer 302a that is proximal to body portion 301 and a second layer 302b that is distal to body portion 301, similar to those described in relation to FIG. 1. FIG. 3 also shows an actuator 303 having a first actuator portion 303a and a second actuator portion 303b that may be compressed toward one another in order to actuate applicator 300 as described herein.

The present disclosure is also directed to methods of using the applicators as described herein. For example, the method may include applying a fluid to a surface via the applicator in order to perform one or more cleaning operations. In one non-limiting example, the one or more cleaning operations according to the present disclosure may include one or more disinfection steps. As used herein, the term “disinfect” means destroying, inactivating, or significantly reducing the concentration of at least a portion of microorganisms present on an inanimate surface and/or reducing or preventing the growth of microorganisms on an inanimate surface. Example inanimate surfaces include, but are not limited, work surfaces in a medical setting, surfaces of medical devices, and combinations thereof. Additionally, or alternatively, the one or more cleaning operations according to the present disclosure may include one or more antiseptic action steps. As used herein, performing an “antiseptic action” means destroying, inactivating, or significantly reducing the concentration of at least a portion of microorganisms present on a human or animal surface and/or reducing or preventing the growth of microorganisms on a human or animal surface. Example human and animal surfaces include, but are not limited to, skin, wound surfaces, hair follicles, mucous membranes, and combinations thereof.

While the aspects described herein have been described in conjunction with the example aspects outlined above, various alternatives, modifications, variations, improvements, and/or substantial equivalents, whether known or that are or may be presently unforeseen, may become apparent to those having at least ordinary skill in the art. Accordingly, the example aspects, as set forth above, are intended to be illustrative, not limiting. Various changes may be made without departing from the spirit and scope of the disclosure. Therefore, the disclosure is intended to embrace all known or later-developed alternatives, modifications, variations, improvements, and/or substantial equivalents.

Thus, the claims are not intended to be limited to the aspects shown herein, but are to be accorded the full scope consistent with the language of the claims, wherein reference to an element in the singular is not intended to mean “one and only one” unless specifically so stated, but rather “one or more.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed as a means plus function unless the element is expressly recited using the phrase “means for.”Herein, the recitation of numerical ranges by endpoints (e.g. between about 50:1 and 1:1, between about 100 and 500° C., between about 1 minute and 60 minutes) include all numbers subsumed within that range, for example, between about 1 minute and 60 minutes includes 21, 22, 23, and 24 minutes as endpoints within the specified range. Thus, for example, ranges 22-36, 25-32, 23-29, etc. are also ranges with endpoints subsumed within the range 1-60 depending on the starting materials used, temperature, specific applications, specific embodiments, or limitations of the claims if needed. The Examples and methods disclosed herein demonstrate the recited ranges subsume every point within the ranges because different synthetic products result from changing one or more reaction parameters. Further, the methods and Examples disclosed herein describe various aspects of the disclosed ranges and the effects if the ranges are changed individually or in combination with other recited ranges.

Further, the word “example” is used herein to mean “serving as an example, instance, or illustration.” Any aspect described herein as “example” is not necessarily to be construed as preferred or advantageous over other aspects. Unless specifically stated otherwise, the term “some” refers to one or more. Combinations such as “at least one of A, B, or C,” “at least one of A, B, and C,” and “A, B, C, or any combination thereof” include any combination of A, B, and/or C, and may include multiples of A, multiples of B, or multiples of C. Specifically, combinations such as “at least one of A, B, or C,” “at least one of A, B, and C,” and “A, B, C, or any combination thereof” may be A only, B only, C only, A and B, A and C, B and C, or A and B and C, where any such combinations may contain one or more member or members of A, B, or C. Nothing disclosed herein is intended to be dedicated to the public regardless of whether such disclosure is explicitly recited in the claims.

As used herein, the term “about” and “approximately” are defined to being close to as understood by one of ordinary skill in the art. In one non-limiting embodiment, the term “about” and “approximately” are defined to be within 10%, preferably within 5%, more preferably within 1%, and most preferably within 0.5%.

What is claimed is:

1. An applicator comprising:
a body portion housing a fluid; and
an application member comprising a tinting agent releasably immobilized therein and/or thereon,
wherein the applicator is free of tinting agent within the body portion.

2. The applicator of claim 1, wherein the application member comprises:
a tinting portion comprising the tinting agent; and
a non-tinting portion, wherein the non-tinting portion of free of tinting agent.

3. The applicator of claim 2, wherein the tinting portion is defined by an interior boundary that is spaced from an outer edge of the application member.

4. The applicator of claim 3, wherein the tinting portion is a continuous region.

5. The applicator of claim 3, wherein the tinting portion comprises more than one discrete regions.

6. The applicator of claim 1, wherein the fluid is contained within an ampoule housed by the body portion, and wherein the applicator further comprises a fluid metering device within the body portion.

7. The applicator of claim 1, wherein the application member comprises an open-celled foam material.

8. The applicator of claim 1, wherein the application member comprises a first layer positioned away from the body portion and a second layer positioned towards the body portion, wherein the first layer comprises the tinting agent and wherein the second layer is free of tinting agent.

9. The applicator of claim 8, wherein the first layer comprises an open-celled foam material and the second layer comprises a non-woven material.

10. The applicator of claim 1, wherein the fluid is a cleaning fluid.

11. The applicator of claim 10, wherein the cleaning fluid comprises an antiseptic selected from the group consisting of octenidine dihydrochloride, chlorhexidine gluconate, and a combination thereof.

12. The applicator of claim 1, wherein all outer edges of the application member are free of tinting agent.

13. A method of making an application member comprising:
providing an application member material; and
applying a tinting agent to a tinting portion of the application member material via a controllable tinting process sufficient to releasably immobilize the tinting agent on and/or in the application member material.

14. The method of claim 13, wherein the tinting agent is comprised by a tinting portion of the application member, wherein the tinting portion is defined by an interior boundary that is spaced from an outer edge of the application member.

15. The method of claim 14, wherein the application member comprises a non-tinting portion, wherein the non-tinting portion of free of tinting agent.

16. The method of claim 13, wherein the controllable tinting process comprises a spraying step.

17. The method of claim 13, wherein the application member material comprises an open-celled foam material.

18. The method of claim 13, further comprising forming a first layer of the application member from the application member material and providing a second layer of the application member.

19. The method of claim 18, wherein the second layer is free of tinting agent.

20. An application member comprising an application member material and a tinting agent releasably immobilized therein and/or thereon,
wherein the tinting agent is comprised by a tinting portion of the application member, and
wherein the tinting portion is defined by an interior boundary that is spaced from an outer edge of the application member.
